(19) United States
(12) Patent Application Publication
(10) Pub. No.: US 2026/0034120 A1
(43) Pub. Date: Feb. 5, 2026

(54) TREATING CANCER WITH LONG-ACTING TOPOISOMERASE I INHIBITOR

(71) Applicant: ProLynx LLC, San Francisco, CA (US)

(72) Inventors: Daniel V. SANTI, San Francisco, CA (US); Shaun D. FONTAINE, Concord, CA (US); Gary W. ASHLEY, Alameda, CA (US)

(73) Assignee: ProLynx LLC, San Francisco, CA (US)

(21) Appl. No.: 19/101,241

(22) PCT Filed: Aug. 3, 2023
(86) PCT No.: PCT/US2023/071592

§ 371 Date: Feb. 4, 2025

Related U.S. Application Data
(60) Provisional application No. 63/370,476, filed on Aug. 4, 2022.

(51) Int. Cl. A61K 31/4745; A61K 31/497; A61K 31/5025; A61K 31/519; A61K 31/5377; A61K 31/55; A61P 35/00

(52) U.S. Cl. CPC A61K 31/4745; A61K 31/497; A61K 31/5025; A61K 31/519; A61K 31/5377; A61K 31/55; A61P 35/00

(57) ABSTRACT

The disclosure provides method of treating cancer in a patient with ATM-deficient or ATR-deficient tumors, comprising safely and efficaciously administering to the patient PLXO38, a long lasting-PEGylated prodrug of the topoisomerase I inhibitor. The disclosure further provides combination therapies of PLXO38 with inhibitors of the DNA damage response (DDR).

### CROSS-REFERENCE TO RELATED APPLICATIONS

This application claims priority to and the benefit of U.S. Provisional Application No. 63/370,476, filed on Aug. 4, 2022, the disclosure of which is incorporated herein by reference in its entirety.

### FIELD

Provided herein are methods and compositions for treating cancer in a patient with an ATM-deficient or ATR-deficient tumor.

### BACKGROUND

Defective DNA repair is a hallmark of cancer and results in genomic instability and accumulation of other genetic abnormalities. Germline mutations of genes involved in DNA repair, such as ataxia telangiectasia mutated (ATM), breast cancer (BRCA) 1 or 2, and tumor protein 53 (TP53) result in markedly increased susceptibility to a variety of cancers. Somatic mutations in these genes are among the most commonly found aberrations in cancer. ATM plays a central role in DNA damage response (DDR) and is activated by DNA double-strand breaks generated either directly by ionizing radiation or reactive chemicals, or indirectly via the processing of other types of DNA lesions or breakdown of DNA replication forks. ATM phosphorylates and thus activates various proteins that together coordinate the arrest of cell cycle progression and DNA repair pathways to preserve genome integrity. Mutations in ATM are among the most common somatic and hereditary cancer mutations in the general population.

Cancers with defective DNA repair mechanisms are commonly more sensitive to treatments that induce DNA damage. ATM loss causes hypersensitivity to various DNA damaging agents including poly (ADP-ribose) polymerase inhibitors (PARP inhibitor), topoisomerase I inhibitors (TOP1 inhibitor), and other S-phase DNA-damaging agents. ATM deficiency also increases the dependence of cancer cells on complementary DNA repair mechanisms, specifically repair of replication stress that is incurred by dividing cells. Inhibition of ataxia telangiectasia and Rad3-related (ATR), the regulator of replication stress response, lethally sensitizes cells with defective ATM to chemotherapy-induced DNA damage. However, clinical trials of PARP inhibitors in ATM deficient tumors have yielded less impressive results compared to those with BRCA1/2 deficiency. The addition of the PARP inhibitor olaparib to paclitaxel failed to improve overall survival over paclitaxel alone in patients with recurrent gastric cancer with low or absent ATM expression. Bang et al. Lancet Oncol 2017; 18 (12): 1637-51 doi 10.1016/S1470-2045 (17) 30682-4. Also, men with metastatic castration-resistant prostate cancer harboring ATM mutations experienced inferior outcomes to PARP inhibitor therapy compared to those harboring BRCA1/2 mutations. Marshall et al. Eur Urol 2019; 76 (4): 452-8 doi 10.1016/j.cururo.2019.02.002. Thus, novel therapeutic approaches are required to leverage the unique sensitivities of ATM-deficient cancers.

Irinotecan, or CPT-11, is a clinically important topoisomerase I inhibitor (TOP1i) widely used in treatment of some of the most common cancers. CPT-11 is metabolically converted to the active metabolite SN-38 which binds tightly to TOP1 cleavage complexes (TOP1cc), which are cytotoxic by their conversion into DNA damage by replication and transcription fork collisions. However, the SN-38 formed from CPT-11 has a short half-life of ˜12 hours; without constant exposure to SN-38, the TOP1cc-TOP1i rapidly reverses and the inhibition of TOP1 and DNA damage is terminated. Hence, the duration of DNA damage caused by SN-38 is limited by the short in vivo lifetime of the inhibitor. Nevertheless, in a recent Phase 1 trial of the combination of irinotecan and rucaparib, patients with ATM-mutated cancers exhibited most benefit. PLX038 is a long-acting prodrug of SN-38 composed of a 40 kDa PEG attached to 4 SN-38 moieties by linkers that slowly cleave to release SN-38. The prodrug and released SN-38 have t½ values of about 5 days in humans, about 20-fold longer than the SN-38 released from CPT-11. Also, the small 15 nm nano-molecule readily penetrates large pores of tumor vasculature, and accumulates and is retained in the tumor microenvironment for long periods through the enhanced permeability and retention effect. Hence, PLX038 should provide a prolonged duration of DNA damage to achieve synthetic lethality of DNA repair deficient or inhibited tumors.

Because of the well-established synergy of TOP1 inhibitors with PARP inhibitors, and the hypersensitivity of ATM deficient cells to both TOP1 and PARP inhibition, we hypothesized that the combination of both agents might be particularly effective in tumors with ATM loss. However, thus far, the combination of TOP1 inhibitor and a PARPi inhibitor have been unsuccessful because of overlapping myelosuppression from both drug classes. One approach to achieve selective tumor inhibition involves the use of a “gapped-schedule” to administer the Top1 inhibitor and the PARP inhibitor. Specifically, the tumor-targeted TOP1 inhibitor can be administered first, and allowed time (a “gap”) to accumulate in the tumor and clear from the normal tissue. Then, the PARP inhibitor can be introduced to prevent repair of DNA damaged by the Top1 inhibitor. However, the underlying pharmacokinetic profiles of the two agents often precludes the use of a gapped schedule.

Accordingly, there is a need to develop anti-cancer therapies using a TOP1 inhibitor with a PARP inhibitor that are both safe and efficacious. Moreover, there is a need to develop safe and effective anti-cancer therapies using TOP1 inhibitor to treat patients with ATM-deficient or ATR-deficient tumors. Furthermore, there is a need to develop anti-cancer therapies using DNA damaging agents, particularly TOP1 inhibitors and PARP inhibitors, in combination with inhibitors of DNA repair mechanisms, particularly ATM kinase and ATR kinase inhibitors.

### BRIEF SUMMARY

Provided herein are methods and compositions for treating cancer in a patient with an ATM-deficient or ATR-deficient tumor, comprising administering to the patient a 4-armed PEG that has SN-38 attached by a slowly cleavable releasable linker to the ends of each arm. In one embodiment, the 4-armed PEG/SN-38 is PLX038, which is of the above formula below, where m is 1 and n is approximately 225.

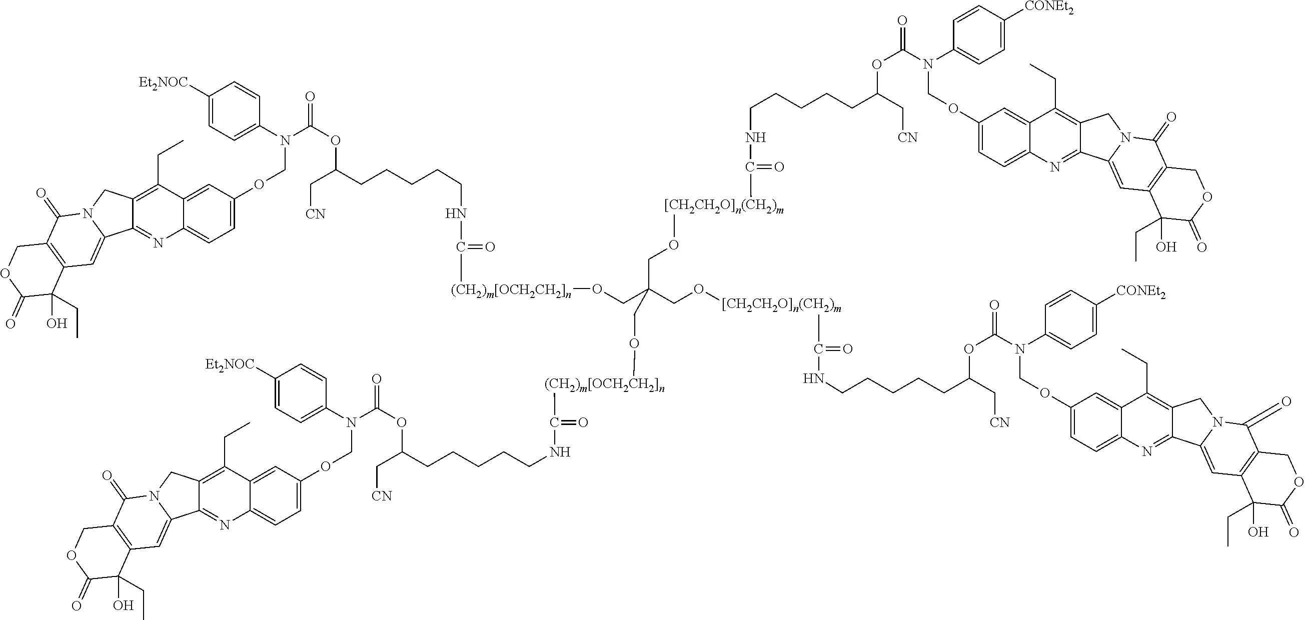

Surprisingly, it has been discovered that PLX038 can be administered to cancer patients with tumors deficient in defective DNA repair mechanisms (e.g., ATM-deficient tumors), at doses that provide high levels of efficacy without significant side effects. Moreover, PLX038 can be administered in combination with a PARP inhibitor to patients with tumors deficient in defective DNA repair mechanisms (e.g., ATM-deficient tumors) under dosing regimens described herein. The combination of the PLX038 and the PARP inhibitor shows remarkable synergy. Importantly, the two agents can be administered at doses that do not compromise efficacy, and therefore overcomes a long-recognized problem with combinations with TOP1 and PARP1 inhibitors.

PLX038 can be administered to patients with ATM-deficient or ATR-deficient tumors at dose levels that provide high levels of SN-38 exposure in the tumor without providing high levels of SN-38 in the plasma. Accordingly, the safety profile of PLX038 is improved relative to other prodrugs of SN-38 such as irinotecan and EZN-2208. Additionally, the improved therapeutic index enables co-administration with other anti-cancer therapies, particularly DNA damaging agents or inhibitors of the DDR.

In one aspect, the disclosure provides a method of treating cancer in a patient with an ATM-deficient or ATR-deficient tumor, comprising administering a parenteral (e.g., intravenous (IV)) dose of PLX038 once every three weeks, wherein the dose provides a steady state AUC_{(0-∞)} of SN-38 from about 2,000 nM·h to about 8,000 nM·h. These steady state exposures of SN-38 can be achieved without providing plasma concentrations of SN-38 generally associated with toxicity. For instance, in some embodiments, PLX038 can be administered intravenously once every three weeks at a dose that provides a steady state Cₘₐₓ of SN-38 less than 100 nM. In other embodiments, PLX038 can be administered intravenously once every three weeks at a dose that provides a steady state Cₘₐₓ of SN-38 from about 30 nM to about 100 nM.

In some embodiments, the dose of PLX038 administered to the cancer patient with an ATM-deficient or ATR-deficient tumor once every three weeks is from about 800 mg/m² to about to about 2.000 mg/m². In other embodiments, the dose of PLX038 administered to the cancer patient once every three weeks is from about 1,000 mg/m² to about 2,000 mg/m². In other embodiments, the dose of PLX038 administered to the cancer patient once every three weeks is from about 1,500 mg/m² to about 2,000 mg/m². In other embodiments, the dose of PLX038 administered to the cancer patient once every three weeks is about 1,730 mg/m².

In another aspect, the disclosure provides a method of treating cancer with an ATM-deficient or ATR-deficient tumor, comprising administering a parenteral (e.g., IV) dose of PLX038 once every three weeks, wherein the dose provides a steady state AUC_{(0-∞)} of SN-38 from about 2,000 nM·h to about 8,000 nM·h, and wherein the PLX038 is administered in combination with a PARP inhibitor (e.g., rucaparib). These steady state exposures of SN-38 can be achieved without providing plasma concentrations of SN-38 generally associated with toxicity. For instance, in some embodiments, PLX038, when administered in combination with a PARP inhibitor, can be administered intravenously once every three weeks at a dose that provides a steady state Cₘₐₓ of less than 50 nM.

In some embodiments, the dose of PLX038 administered once every three weeks to the cancer patient with the ATM-deficient or ATR-deficient tumor, when administered in combination with a PARP inhibitor, is from about 350 mg/m² to about 1,300 mg/m². In OTHER embodiments, the dose of PLX038 administered once every three weeks to the cancer patient with the ATM-deficient or ATR-deficient tumor, when administered in combination with a PARP inhibitor, is from about 600 mg/m² to about 1,200 mg/m². In other embodiments, the dose of PLX038 administered once every three weeks, when administered in combination with a PARP inhibitor, is from about 750 mg/m² to about 1,100 mg/m². It will be understood that the PLX038 and the PARP inhibitor can be administered simultaneously or sequentially. In preferred embodiments, the PARP inhibitor is administered from 2-6 days after the PARP inhibitor in a set dosing schedule. In some embodiments, the PARP inhibitor is administered 5 days after the PLX038 in a set dosing schedule. In one embodiment, PLX038 is administered by IV infusion on day 1 of every 21-day cycle at a dose as set forth herein, and the PARP inhibitor (e.g., rucaparib) is administered orally twice daily on days 5-19 of the cycle.

In some embodiments, the PLX038, either alone or in combination with a PARP inhibitor, is administered to a patient that has breast cancer. In some such embodiments, the patient has triple-negative breast cancer. In other embodiments, the patient has ovarian cancer. In other embodiments, the patient has small cell lung cancer.

In another aspect, the disclosure provides methods of treating cancer in a patient in need thereof, comprising administering the patient a combination of PLX038 and an ATM kinase inhibitor or an ATR kinase inhibitor. In some embodiments, PLX038 and the ATM kinase inhibitor or ATR kinase inhibitor are administered simultaneously. In other embodiments, PLX038 and the ATM kinase inhibitor or ATR kinase inhibitor are administered sequentially. For instance, PLX038 and the ATM kinase inhibitor or ATR kinase inhibitor may be administered according to a gapped schedule. In some embodiments, the ATM kinase inhibitor or ATR kinase inhibitor is administered between 2 days and 5 days after administration of PLX038. In some embodiments, a PARP inhibitor is also administered to the patient.

### BRIEF DESCRIPTION OF FIGURES

FIG. 1 shows anti-tumor effects of PLX038A in 22Rv1WT or 22Rv1 ATM KO xenografts. Mice (N=8/group) bearing 22RV1 xenografts received a single IP dose of vehicle (●) or PLX038A at 7.5 (♦), 15 (▾), 30 (▴) or 60 (▪) μmol/kg. A) Median relative tumor volume over time for mice bearing 22Rv1WT xenografts. B) Event-free survival for animals bearing 22Rv1WT tumors. C) Median relative tumor volume over time for mice bearing 22Rv1 ATM KO xenografts. D) Event-free survival for animals bearing 22Rv1 ATM KO xenografts. In panels B and D an event is a 4-fold increase in tumor volume from that on day 0 of treatment. Following are significance of treatments over 1 month: in panel A, vehicle vs PLX038A at 7.5 was p<0.01 (**), vehicle vs PLX038A at 15 μmol/kg was p<0.001 (***), and vehicle versus PLX038A at 30- or 60 μmol/kg was p<0.0001 (****). In panel C, significance of vehicle vs PLX038A at 7.5-, 15-, 30-, and 60 μmol/kg was p<0.0001 (****). The significance of treatments in the time to 4-fold increase in tumor size were: in panel B, vehicle vs PLX038A at 7.5- was p<0.001 (***), and 15-, 30- and 60 μmol/kg was p<0.0001 (****). In panel D, vehicle vs PLX038A at 7.5-, 15-, 30-, and 60 μmol/kg was p<0.0001 (****). When comparing AUC of growth curves over ˜1 month, 22Rv1 ATM KO xenografts show increased sensitivity over WT at significance p<0.001 (***) for the three lower doses, and p<0.05 (*) at the highest dose.

FIG. 2 shows Anti-tumor efficacy of the combination of PLX038A and TLZ in mice bearing 22Rv1 ATM KO tumors. A) Median relative tumor volume over time of 22Rv1 ATM KO xenografts. B) Event-free survival for 22Rv1 ATM KO xenografts where an event is a 4-fold increase in tumor volume from that on day 0 of treatment. In panel A,B mice (N=5) bearing 22RV1 xenografts were treated with vehicle (●), a single IP dose of PLX038A at 7.5 μmol/kg (▪), QD PO TLZ at 0.4 μmol/kg (▴), or a combination of a single dose IP dose of PLX038A at 7.5 μmol/kg along with QD PO TLZ at 0.4 μmol/kg for the duration of the study (▾). In panel C,D mice (N=5) xenografts were treated as above with 50% the concentrations of PLX038A and TLZ used in panels A and B. In Panel A and C, the data with combinations are significantly different than those of vehicle or single agents (p<0.05, *). The difference between growth with QD 0.2 μmol/kg TLZ and vehicle in panel C is not statistically different. The significance of treatments in the time to 4-fold increase in tumor size were: in panel B, the vehicle vs PLX038A was p<0.01 (**), vehicle vs QD TLZ was p<0.05 (*), PLX038 vs combo was p<0.05 (*), and TLZ vs combo was p<0.01 (**); in panel D, combo vs PLX038A or TLZ was p<0.01 (**).

FIG. 3 shows Tumor growth of 22Rv1 KO tumors after single IP doses of PLX038A, QD TLZ or the combination. A, C, E) Relative tumor volume versus time post-treatment. B,D,F) Event-free survival where an event is defined as a 4-fold increase in tumor volume from day 0. Mice (n=5/group) received a single IP dose of vehicle (●), PLX038A at 3.75 μmol/kg (▪) or 1.9 μmol/kg (▴), QD PO TLZ at 0.3 μmol/kg (Δ) or 0.2 μmol/kg (▾), or combinations of PLX038A at 3.75 and QD PO TLZ at 0.3 μmol/kg (♦), PLX038A at 1.9 μmol/kg and QD PO TLZ at 0.3 μmol/kg (+), or PLX038A at 1.9 μmol/kg and QD PO TLZ at 0.2 μmol/kg (⋄).

FIG. 4 shows response to PLX038 and rucaparib combination in a patient with metastatic breast cancer with pathogenic germline ATM mutation. (A) Fluorodeoxyglucose-positron emission tomography (FDG-PET) before and 2 mo after starting treatment. The recurrent breast cancer lesion in the skin regressed completely anatomically and metabolically (yellow arrows) as well as the metastatic lesions. (B) Lolliplot representing the mutation in the ATM gene.

### DETAILED DESCRIPTION

Provided herein are methods and compositions for treating cancer in a patient with an ATM-deficient or ATR-deficient tumor, comprising administering to the patient a 4-armed PEG that has SN-38 attached by a slowly cleavable releasable linker to the ends of each arm. Also provided are methods of treating cancer in a patient in need thereof, comprising administering a 4-armed PEG that has SN-38 attached by a slowly cleavable releasable linker to the ends of each arm in combination with an ATM kinase inhibitor or ATR kinase inhibitor.

In one embodiment, the 4-armed PEG has the following structure:

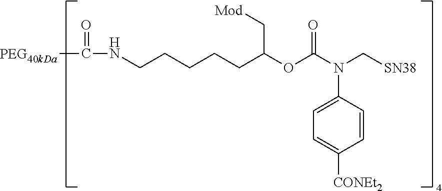

In one embodiment, the 4-armed PEG has the following structure:

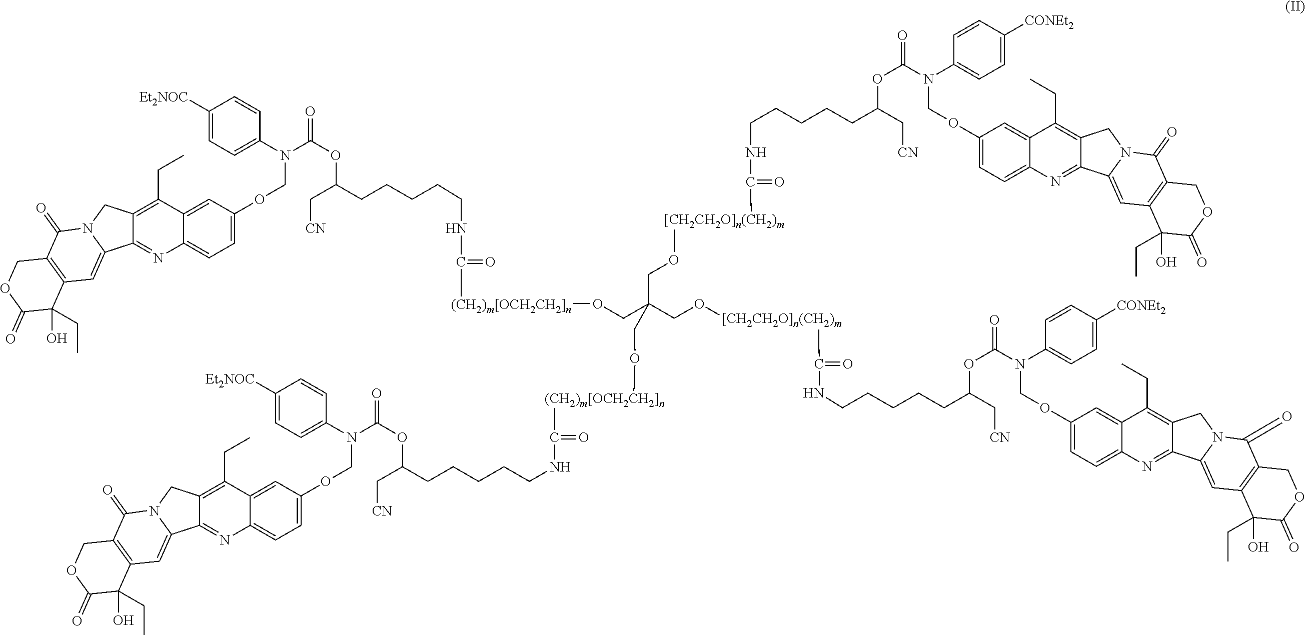

wherein m=1-6 and n is 200-250. In particular embodiments, the conjugate may be PLX038, which is of the above formula where m is 1 and n is approximately 225. PLX038 is disclosed in U.S. Pat. No. 10,016,411, the content of which is incorporated by reference in its entirety. The chemical name for PLX-038 is O-[7-(tetra-polyethylene glycol ether)-carboxamido-1-cyano-2-heptyl]-N-(10′-methoxy-7′-ethyl-camptothecin)-N-[4′-(diethylcarbamoyl)phenyl)]carbamate. In other embodiments, the 4-armed PEG has Mod=CH₃SO₂ and is PLX038A.

In one aspect, the disclosure provides a method of treating cancer in a patient with an ATM-deficient or ATR-deficient tumor, comprising administering a parenteral (e.g., intravenous (IV)) dose of PLX038 to the patient once every three weeks, wherein the dose provides a steady state AUC_{(0-∞)} of SN-38 from about 2,000 nM·h to about 8,000 nM·h. In some embodiments, the dose of PLX038 once every three weeks provides a steady state AUC_{(0-∞)} of SN-38 from about 3,500 nM·h to about 7,500 nM·h. In some embodiments, the parenteral (e.g., IV) dose of PLX038 once every three weeks provides a steady state AUC_{(0-∞)} of SN-38 from about 4,000 nM·h to about 7,000 nM·h. In some embodiments, the parenteral (e.g.) IV dose of PLX038 once every three weeks provides a steady state AUC_{(0-∞)} of SN-38 from about 4,500 nM·h to 6,500 about nM·h. In some embodiments, the parenteral (e.g., IV) dose of PLX038 once every three weeks provides a steady state AUC_{(0-∞)} of SN-38 from about 5,000 nM·h to about 6,500 nM·h. In some embodiments, the parenteral (e.g., IV) dose of PLX038 once every three weeks provides a steady state AUC_{(0-∞)} of SN-38 from about 5,500 nM·h to about 6,400 nM·h.

The particular steady state exposures of SN-38 set forth above (e.g., about 2,000 nM·h to about 8,000 nM·h, about 3,500 nM·h to about 7,500 nM·h, about 4,000 nM·h to about 7,000 nM·h., about 4,500 nM·h to about 6,500 nM·h, or about 5,000 nM·h to about 6,500 nM·h) can be achieved without providing plasma concentrations of SN-38 generally associated with toxicity. For instance, in some embodiments, PLX038 can be administered parenterally (e.g., intravenously) once every three weeks at a dose that provides a steady state Cₘₐₓ of SN-38 of less than 100 nM. In other embodiments, PLX038 can be administered intravenously once every three weeks at a dose that provides a steady state Cₘₐₓ of less than 80 nM. In other embodiments, PLX038 can be administered parenterally (e.g., intravenously) once every three weeks at a dose that provides a steady state Cₘₐₓ of SN-38 of less than 70 nM. In other embodiments, PLX038 can be administered parenterally (e.g., intravenously) once every three weeks at a dose that provides a steady state Cₘₐₓ of SN-38 of less than 50 nM. In other embodiments, PLX038 can be administered parenterally (e.g., intravenously) once every three weeks at a dose that provides a steady state Cₘₐₓ of SN-38 of from about 30 nM to about 100 nM. In other embodiments, PLX038 can be administered parenterally (e.g., intravenously) once every three weeks at a dose that provides a steady state Cₘₐₓ of SN-38 of from about 40 nM to about 90 nM. In other embodiments, PLX038 can be administered parenterally (e.g., intravenously) once every three weeks at a dose that provides a steady state Cₘₐₓ of from about 45 nM to about 85 nM. In other embodiments, PLX038 can be administered parenterally (e.g., intravenously) once every three weeks at a dose that provides a steady state Cₘₐₓ of from about 50 nM to about 75 nM. In other embodiments, PLX038 can be administered parenterally (e.g., intravenously) once every three weeks at a dose that provides a steady state Cₘₐₓ of from about 30 nM to about 65 nM. In other embodiments, PLX038 can be administered parenterally (e.g., intravenously) once every three weeks at a dose that provides a steady state Cₘₐₓ of SN-38 of from about 35 nM to about 55 nM. In other embodiments, PLX038 can be administered parenterally (e.g., intravenously) once every three weeks at a dose that provides a steady state Cₘₐₓ of SN-38 of from about 40 nM to about 50 nM.

In some embodiments, the dose of PLX038 once every three weeks is from about 350 mg/m² to about to about 2,000 mg/m². In other embodiments, the dose of PLX038 once every three weeks is from about 1,000 mg/m² to about 2,000 mg/m². In other embodiments, the dose of PLX038 once every three weeks is from about 1,500 mg/m² to about 2,000 mg/m². In other embodiments, the dose of PLX038 once every three weeks is from about 1,500 mg/m² to about 1,800 mg/m². In other embodiments, the dose of PLX038 once every three weeks is from about 1,500 mg/m² to about 1,700 mg/m². In other embodiments, the dose of PLX038 once every three weeks is about 1,000 mg/m². In other embodiments, the dose of PLX038 once every three weeks is about 1,500 mg/m². In other embodiments, the dose of PLX038 once every three weeks is about 1,700 mg/m². In other embodiments, the dose of PLX038 once every three weeks is about 1,730 mg/m². In other embodiments, the dose of PLX038 once every three weeks is about 1,750 mg/m². In other embodiments, the dose of PLX038 once every three weeks is about 1,850 mg/m².

In another aspect, the disclosure provides a method of treating cancer in a patient with an ATM-deficient or ATR-deficient tumor, comprising administering a parenteral (e.g., intravenous) dose of PLX038 once every three weeks, wherein the dose provides a steady state AUC_{(0-∞)} of SN-38 from about 2,000 nM·h to about 6,000 nM·h, and wherein the PLX038 is administered in combination with a PARP inhibitor. In some embodiments, the PLX038, when administered in combination with a PARP inhibitor, the PLX038 is administered at an IV dose once every three weeks, wherein the dose provides a steady state AUC_{(0-∞)} of SN-38 from about 2,000 nM·h to about 4,500 nM·h. In other embodiments, the PLX038, when administered in combination with a PARP inhibitor, is administered at an IV dose once every three weeks, wherein the dose provides a steady state AUC_{(0-∞)} of SN-38 from about 2,500 nM·h to about 4,000 nM·h. In other embodiments, the PLX038, when administered in combination with a PARP inhibitor, is administered at an IV dose once every three weeks, wherein the dose provides a steady state AUC_{(0-∞)} of SN-38 from about 2,500 nM·h to about 3,500 nM·h. In other embodiments, the PLX038, when administered in combination with a PARP inhibitor, is administered at an IV dose once every three weeks, wherein the dose provides a steady state AUC_{(0-∞)} of SN-38 from about 2,700 nM·h to about 3,700 nM·h. In some such embodiments, the PARP inhibitor is rucaparib. In other such embodiments, the PARP inhibitor is olaparib. In other such embodiments, the PARP inhibitor is niraparib. In other such embodiments, the PARP inhibitor is talazoparib. In some embodiments, the DDR inhibitor is administered after a period of time in which the PLX038 accumulates in the tumor. For instance, the DDR inhibitor may be administered from two to six days following administration of PLX038. In certain embodiments, the DDR inhibitor is administered 4 days after following administration of PLX038.

The particular steady state exposures of SN-38 set forth above (e.g., about 2,000 nM·h to about 6,000 nM·h, about 2,000 nM·h to about 4,500 nM·h, about 2,500 nM·h to about 4,000 nM·h., about 2,500 nM·h to 3,500 about nM·h, or about 2,700 nM·h to about 3,700 nM·h) when PLX-38 is administered as part of a combination therapy with a PARP inhibitor can be achieved without providing plasma concentrations of SN-38 generally associated with toxicity. For instance, in some embodiments, PLX038, when administered in combination with a PARP inhibitor, can be administered intravenously once every three weeks at a dose that provides a steady state Cₘₐₓ of less than 50 nM. In other embodiments, PLX038, when administered in combination with a PARP inhibitor, can be administered intravenously once every three weeks at a dose that provides a steady state Cₘₐₓ of less than 40 nM. In other embodiments, PLX038, when administered in combination with a PARP inhibitor, can be administered intravenously once every three weeks at a dose that provides a steady state Cₘₐₓ of less than 30 nM. In other embodiments, PLX038, when administered in combination with a PARP inhibitor, can be administered intravenously once every three weeks at a dose that provides a steady state Cₘₐₓ of less than 20 nM. In other embodiments, PLX038, when administered in combination with a PARP inhibitor, can be administered intravenously once every three weeks at a dose that provides a steady state Cₘₐₓ of from about 15 nM to about 50 nM. In other embodiments, PLX038, when administered in combination with a PARP inhibitor, can be administered intravenously once every three weeks at a dose that provides a steady state Cₘₐₓ of from about 20 nM to about 40 nM. In other embodiments, PLX038, when administered in combination with a PARP inhibitor, can be administered intravenously once every three weeks at a dose that provides a steady state Cₘₐₓ of from about 20 nM to about 30 nM.

In some embodiments, the dose of PLX038 once every three weeks, when administered in combination with a PARP inhibitor, is from about 350 mg/m² to about 1,300 mg/m². In other embodiments, the dose of PLX038 once every three weeks, when administered in combination with a PARP inhibitor, is from about 750 mg/m² to about 1,100 mg/m². In other embodiments, the dose of PLX038 once every three weeks, when administered in combination with a PARP inhibitor, is from about 800 mg/m² to about 1,000 mg/m². In other embodiments, the dose of PLX038 once every three weeks, when administered in combination with a PARP inhibitor, is about 800 mg/m². In other embodiments, the dose of PLX038 once every three weeks, when administered in combination with a PARP inhibitor, is about 900 mg/m². In other embodiments, the dose of PLX038 once every three weeks, when administered in combination with a PARP inhibitor, is about 1,000 mg/m². In other embodiments, the dose of PLX038 once every three weeks, when administered in combination with a PARP inhibitor, is about 1,100 mg/m².

In some embodiments where PLX038 is administered in combination with a PARP inhibitor (e.g., rucaparib), the PARP inhibitor is administered each cycle only after the PLX038 sufficiently accumulates in the tumor of the cancer patient and is depleted in the plasma of the patient. In one such embodiment, PLX038 is administered by IV infusion on day 1 of every 21-day cycle using a dosing regimen as set forth herein, and rucaparib is administered orally twice daily on days 5-19 of the cycle. In some embodiments, the rucaparib is administered at a dose of from about 300 mg to about 600 mg (e.g., about 300 mg, about 400 mg or about 600 mg) twice daily.

In some embodiments, a parenteral (e.g., intravenous) dose of PLX038 of about 1,300 g/m² is administered to the patient on day 1 of every 21 day cycle and an oral dose of about 300 mg of rucaparib is administered orally twice daily to the patient. In other embodiments, a parenteral (e.g., intravenous) dose of about 1,300 g/m² of PLX038 is administered to the patient on day 1 of every 21 day cycle and an oral dose of about 400 mg of rucaparib is administered orally twice daily to the patient. In other embodiments, a parenteral (e.g., intravenous) dose of PLX038 of about 1,300 g/m² is administered to the patient on day 1 of every 21 day cycle and an oral dose of about 600 mg of rucaparib is administered orally twice daily to the patient. In other embodiments, parenteral (e.g., intravenous) dose of about 1,000 g/m² of PLX038 is administered to the patient on day 1 of every 21 day cycle and an oral dose of about 300 mg of rucaparib is administered orally twice daily to the patient. In other embodiments, a parenteral (e.g., intravenous) dose of about 1,000 g/m² of PLX038 is administered to the patient on day 1 of every 21 day cycle and an oral dose of about 400 mg of rucaparib is administered orally twice daily to the patient. In other embodiments, a parenteral (e.g., intravenous) dose of about 1,000 g/m² of PLX038 is administered to the patient on day 1 of every 21 day cycle and an oral dose of about 600 mg of rucaparib is administered orally twice daily to the patient. In other embodiments, a parenteral (e.g., intravenous) dose of about 850 g/m² of PLX038 is administered to the patient on day 1 of every 21 day cycle and an oral dose of about 300 mg of rucaparib is administered orally twice daily to the patient. In other embodiments, a parenteral (e.g., intravenous) dose of about 850 g/m² of PLX038 is administered to the patient on day 1 of every 21 day cycle and an oral dose of about 400 mg of rucaparib is administered orally twice daily to the patient. In other embodiments, a parenteral (e.g., intravenous) dose of about 850 g/m² of PLX038 is administered to the patient on day 1 of every 21 day cycle and an oral dose of about 600 mg of rucaparib is administered orally twice daily to the patient.

In another aspect, the disclosure provides methods of treating cancer in a patient in need thereof, comprising administering the patient a combination of PLX038 and an ATM kinase inhibitor or an ATR kinase inhibitor. In some such embodiments, the patient is deficient in one or more genes that express proteins involved in DNA repair. These genes include, but are not limited to, BRCA1, BRCA2, ATM and ATR. In some embodiments, the patient is not deficient any genes that express proteins involved in DNA repair.

In some embodiments, PLX038 and the ATM kinase inhibitor or ATR kinase inhibitor are administered simultaneously. In other embodiments, PLX038 and the ATM kinase inhibitor or ATR kinase inhibitor are administered sequentially. For instance, PLX038 and the ATM kinase inhibitor or ATR kinase inhibitor may be administered according to a gapped schedule. In some embodiments, the ATM kinase inhibitor or ATR kinase inhibitor is administered between 2 days and 5 days after administration of PLX038. In some embodiments, a PARP inhibitor (e.g., rucaparib) is also administered to the patient.

In some embodiments, the dose of PLX038 once every three weeks, when administered in combination with an ATM kinase inhibitor or ATR kinase inhibitor, is from about 350 mg/m² to about 1.300 mg/m². In other embodiments, the dose of PLX038 once every three weeks, when administered in combination with an ATM kinase inhibitor or ATR kinase, is from about 750 mg/m² to about 1,100 mg/m². In other embodiments, the dose of PLX038 once every three weeks, when administered in combination with a an ATM kinase inhibitor or ATR kinase, is from about 800 mg/m² to about 1,000 mg/m². In other embodiments, the dose of PLX038 once every three weeks, when administered in combination with an ATM kinase inhibitor or ATR kinase, is about 800 mg/m². In other embodiments, the dose of PLX038 once every three weeks, when administered in combination with an ATM kinase inhibitor or ATR kinase, is about 900 mg/m². In other embodiments, the dose of PLX038 once every three weeks, when administered in combination with an ATM kinase inhibitor or ATR kinase, is about 1,000 mg/m². In other embodiments, the dose of PLX038 once every three weeks, when administered in combination with an ATM kinase inhibitor or ATR kinase, is about 1,100 mg/m².

In any of the foregoing embodiments, the PLX038 and ATM kinase inhibitor or ATR kinase can be administered in combination with a PARP inhibitor. In some embodiments, the PLX038 and PARP inhibitor are administered according to a gapped dosing schedule as set forth herein. In some embodiments, the ATM kinase inhibitor or ATR kinase and the PARP inhibitor are both administered between 2 days and 6 days after administration of PLX038. In some such embodiments, the PARP inhibitor and the ATM kinase inhibitor or ATR kinase are administered on the same day. In some such embodiments, the PARP inhibitor and the ATM kinase inhibitor or ATR kinase are administered on different days. In some embodiments, the ATM kinase inhibitor or ATR kinase and the PARP inhibitor are both administered between 4 days after administration of PLX038.

In some embodiments the ATM kinase inhibitor administered in combination with PLX038 is AZD0156, LY294002, KU-55933, or KU-59403.

In some embodiments the ATR kinase inhibitor administered in combination with PLX038 is AZD6738, M6620 (VX-970), BAY1895344 or M4344 (VX-803).

In some embodiments the cancer to be treated is a solid tumor. In some embodiments the cancer is any of adult and pediatric oncology, myxoid and round cell carcinoma, locally advanced tumors, metastatic cancer, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma lung cancer, including small cell carcinoma, cutaneous T cell lymphoma, Hodgkin's lymphoma, non-Hodgkin's lymphoma, cancer of the adrenal cortex, ACTH-producing tumors, nonsmall cell cancers, breast cancer, including small cell carcinoma and ductal carcinoma, gastrointestinal cancers, including stomach cancer, colon cancer, colorectal cancer, polyps associated with colorectal neoplasia, pancreatic cancer, liver cancer, urological cancers, including bladder cancer, including primary superficial bladder tumors, invasive transitional cell carcinoma of the bladder, and muscle-invasive bladder cancer, prostate cancer, malignancies of the female genital tract, including ovarian carcinoma, primary peritoneal epithelial neoplasms, cervical carcinoma, uterine endometrial cancers, vaginal cancer, cancer of the vulva, uterine cancer and solid tumors in the ovarian follicle, malignancies of the male genital tract, including testicular cancer and penile cancer, kidney cancer, including renal cell carcinoma, brain cancer, including intrinsic brain tumors, neuroblastoma, astrocytic brain tumors, gliomas, metastatic tumor cell invasion in the central nervous system, bone cancers, including osteomas and osteosarcomas, skin cancers, including melanoma, tumor progression of human skin keratinocytes, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma. In some such embodiments, PLX038 can be administered to a cancer patient that has a genetic defect in a DDR.

In some embodiments, the PLX038, either alone or in combination with a PARP inhibitor, is administered to a patient that has breast cancer. In some such embodiments, the patient has triple-negative breast cancer. In other embodiments, the patient has ovarian cancer. In other embodiments, the patient has small cell lung cancer. In some such embodiments, PLX038 can be administered to a cancer patient that has a genetic defect in a DDR.

In some embodiments, PLX038 can be administered parenterally to the patient. In some embodiments, the route of administration is intravenous, intra-arterial, intramuscular, or subcutaneous.

The disclosure also provides methods for identifying cancer patients with ATM-deficient tumors and administering PLX038 (either alone or in combination with a PARP inhibitor) to patient with at ATM-deficiency. Identifying loss of ATM function in tumor cells might allow for the characterization of a patient subset that could receive benefit from the approach described herein. The large size of the ATM gene, 66 exons spanning approximately 150 kb of genomic DNA, together with the diversity and broad distribution of mutations renders routine DNA sequencing a challenging diagnostic tool. But cancer-associated ATM mutations can lead to a reduction in ATM protein expression and loss of ATM activity is often associated with reduced ATM protein levels. In addition to deleterious mutations, loss of ATM activity may also result from epigenetic silencing. Thus ATM protein expression by immunohistochemistry may be a valuable clinical tool to identify the patient subgroup spanning multiple tumor types with low or absent ATM protein levels.

### EXAMPLES

### Example 1: a Single Dose of PLX038A is Highly Effective in ATM-Deficient Tumors

### Materials and Methods

Dosing solutions of PLX038A were prepared in isotonic acetate, pH 5, to contain 0.19- to 6 mM of SN-38 to deliver 1.9- to 60 μmol/kg. SN-38 content was verified by A_{363 nm} (ε=22,500 M⁻¹ cm⁻¹). PLX038A is a pharmacokinetic equivalent of PLX-38 for mice. Fontaine et al. Cancer Chemother Pharmacol 2019; 84 (4): 729-38 doi 10.1007/s00280-019-03903-5; Santi et al. Journal of medicinal chemistry 2014; 57 (6): 2303-14 doi 10.1021/jm401644v.

Animal studies were carried out as previously described. Fontaine et al. Cancer Chemother Pharmacol 2019; 84 (4): 729-38 doi 10.1007/s00280-019-03903-5. Animal studies were carried out in accordance with UCSF Institutional Animal Care and Use Committee protocols. MX-1 cells were obtained from the NCI. MX-1 xenografts were established in female nu/nu nude mice as previously reported. Fontaine et al. Cancer Chemother Pharmacol 2019; 84 (4): 729-38 doi 10.1007/s00280-019-03903-5; Morton and Houghton Nat Protoc 2007; 2 (2): 247-50 doi 10.1038/nprot.2007.25. When tumors reached 170 mm³, mice received a single intraperitoneal dose of vehicle, a single intraperitoneal dose of PLX038A (15 μmol/kg).

The ATM 22Rv1 KO, derived via CRISPR/Cas9 genome editing, harbors a biallelic 33-nucleotide insertion in ATM resulting in a premature stop codon; the ATM 22Rv1 KO lacked detectable ATM protein by immunoblotting and IHC. Kaur et al. Clin Cancer Res 2020; 26 (18): 4869-81 doi 10.1158/1078-0432.CCR-20-0764. To establish 22Rv1 xenografts, 10-7 cells (in 100 μL of 1:1 PBS: Matrigel (BD 356237) were subcutaneously injected into the flank of male NSG mice. When tumors reached ˜125 mm, mice received a single intraperitoneal dose of PLX038A (3.75- to 60 μmol/kg) for 21 days. Tumor volumes measured by caliper [0.5×(length×width²)] and body weights were determined twice weekly. Event-free survival analyses were performed using Prism 8.0 with an event defined as a four-fold increase in tumor volume from the day of treatment

### Results

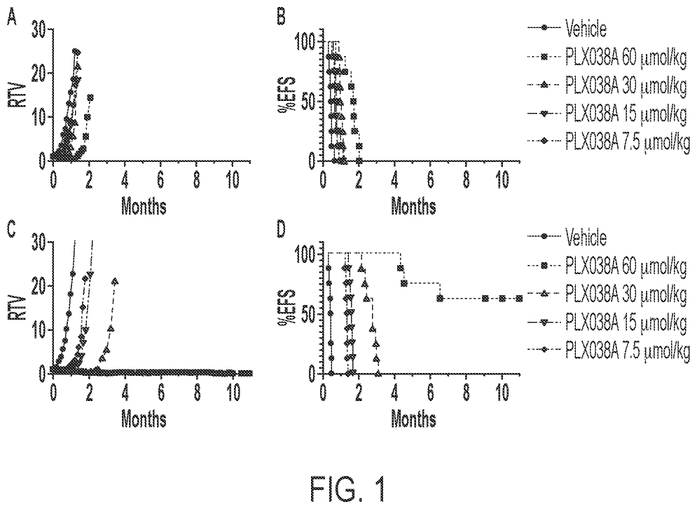

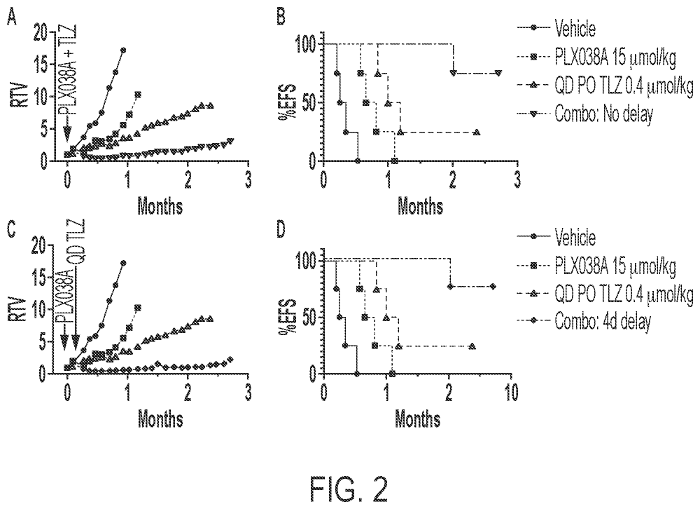

The efficacy of PLX038A as a single agent in an ATM-proficient and deficient tumors was examined. For this study tumor xenografts were formed with an isogenic pair of WT and ATM knockout (ATM KO) 22Rv1 prostate cancer cells, animals were treated with a single intraperitoneal (IP) injection of PLX038A or vehicle control, and monitored tumor growth (FIG. 2). For both the 22Rv1WT and 22Rv1 ATM KO tumors, dose-dependent response in relative tumor volume and event free survival (EFS) was observed, with 22Rv1 ATM KO tumors displaying exquisite sensitivity to PLX038A compared to WT with no significant change in body weight. In the untreated cohorts, the median EFS of animals with 22Rv1WT and 22Rv1 ATM KO tumors was similar at ˜0.5 mo. Doses of PLX038A up to 30 μmol/kg consistently showed 4-fold higher sensitivity of 22Rv1 ATM KO than WT (FIG. 1B, C). A t the highest dose of PLX038A tested (60 μmol/kg), the median EFS of mice bearing 22Rv1WT tumors was 1.7 mo (FIG. 1B) with half of the tumors quadrupling their volume in 54 days, ˜4-fold increase compared to the vehicle control. In contrast, at the same dose of PLX038A, the median EFS of animals with 22Rv1 ATM KO tumors (FIG. 1D) was >11 mo, over a 22-fold increase compared to the 0.5 mo EFS of vehicle control. Moreover, 22Rv1 ATM KO tumors shrank to 10- to 20% of their initial volume over 3 mo. Then, between 4- to 6 months, 3 of 8 of the ATM mutant tumors resumed growth while the remaining 5 were suppressed for an additional 8 mo (FIG. 1C), with no palpable tumors detected in 4 animals. Of the 8 mice treated at 60 μmol/kg PLX038A, 5 survived for 9 mo and 3 until study termination at 11 mo. Overall, from relative increases in median EFS, at 60 μmol/kg PLX038 the ATM KO was over 6-fold more sensitive than the isogenic ATM WT tumor.

### Example 2: PLX038A and a PARP Inhibitor are Highly Effective in ATM-Deficient Tumors

### Materials and Methods

Dosing solutions of PLX038A was prepared as in Example 1. The PARP inhibitor talazoparib (TLZ) was purchased from MedKoo (Cat #204710) and used as received.

Animal studies were carried out as in Example 1. When tumors reached 170 mm³, mice received a single intraperitoneal dose of vehicle, a single intraperitoneal dose of PLX038A (15 μmol/kg), a daily oral dose of talazoparib (0.4 μmol/kg) or a combination of PLX038A (15 μmol/kg) and daily oral TLZ (0.4 μmol/kg) starting on either day 0 (same day as PLX038A dosing) or day 4.

### Results

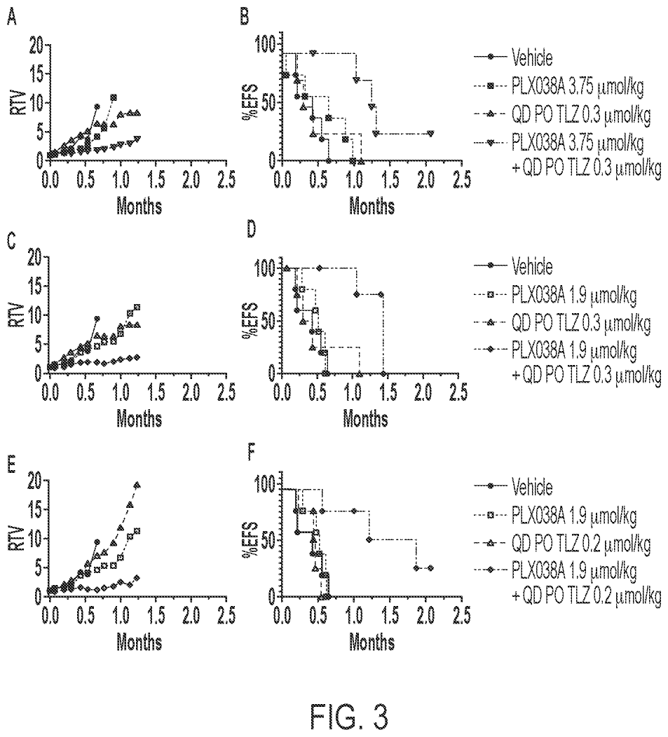

FIG. 2A shows tumor growth of 22Rv1 ATM KO xenografts treated with a single low dose of 7.5 μmol/kg PLX038, daily doses of 0.4 μmol/kg TLZ and a combination of both, and FIG. 2B provides the corresponding Kaplan-Meier plot showing the time for tumors to reach 4-fold their original volume. FIGS. 2C and D shows the same except the drug concentrations used are a low 50% of those in FIGS. 2A,B. TLZ is modestly inhibitory to growth at the higher 0.4 μmol/kg dose (FIG. 2A) and insignificantly inhibitory at the lower 0.2 μmol/kg dose (FIG. 3A). However, both combination doses-even very low doses that give little or no single-agent inhibition-cause long-term growth suppression of the 22RV1 ATM KO and this is clearly supra-additive over the effects calculated from the individual components. The combination of PLX038A and TLZ was tolerated and synergistic at all doses evaluated (Table 1) and was effective at doses down to 1.9 μmol/kg and 0.2 μmol/kg, respectively (FIG. 3). Hence, the hypersensitivity of the 22RV1 ATM KO towards PLX038A is not observed with single agent TLZ, but it is clearly manifest as synergy at even very low doses of the PLX038A-PARPi combination that are barely- or non-inhibitory when administered as single agents.

TABLE 1
Synergy of varying doses of PLX038 and TLZ in ATM KO xenografts

| | MX-1 | 22RV1 ATM KO | 22RV1 ATM KO | 22RV1 ATM KO | 22RV1 ATM KO | 22RV1 ATM KO |
|---|---|---|---|---|---|---|
| T/C_{3 wk} | PLX038A: | PLX038A: | PLX038A: | PLX038A: | PLX038A: | PLX038A: |
| | 15 μmol/kg | 7.5μmol/kg | 3.75 μmol/kg | 3.75μmol/kg | 1.95 μmol/kg | 1.9 μmol/kg |
| | TLZ: 0.4 | TLZ: 0.4 | TLZ: 0.3 | TLZ: 0.2 | TLZ: 0.3 | TLZ: 0.2 |
| | μmol/kg/day | μmol/kg/day | μmol/kg/day | μmol/kg/day | μmol/kg/day | μmol/kg/day |
| Vehicle | 1.0 | 1.0 | 1.0 | 1.0 | 1.0 | 1.0 |
| PLX038A | 0.30 | 0.40 | 0.45 | 0.45 | 0.49 | 0.49 |
| TLZ | 0.21 | 0.72 | 0.69 | 0.76 | 0.69 | 0.76 |
| Combination | 0.038 | 0.17 | 0.20 | 0.20 | 0.20 | 0.12 |
| observed | | | | | | |
| Combination | 0.062 | 0.29 | 0.31 | 0.34 | 0.34 | 0.37 |
| predicted | | | | | | |
| predicted/ | 1.6 | 1.6 | 1.6 | 1.7 | 1.7 | 3.0 |
| observed >1 = | | | | | | |
| synergy | | | | | | |

T/C_{3 wk} is defined as (RTV after treatment for 3 weeks)/(RTV vehicle at 3 weeks).
Combination predicted = T/C_{PLX038A, 3 wk} * T/C_{TLZ, 3 wk}; combination observed = T/C_{combo, 3 wk}

### Example 3: PLX038 and Rucaparib in a Patient with ATM Deficient Breast Cancer

The demonstration of enhanced efficacy of PLX038-PARPi combination in DDR-deficient preclinical models provided compelling rationale to investigate this combination in the clinic. A patient with metastatic breast cancer and germline ATM mutation was enrolled in the ongoing dose escalation phase of the trial. The primary objective of the trial (Clinicaltrials.gov identifier: NCT04209595) is to identify the maximum tolerated dose of PLX038 in combination with PARP inhibitor rucaparib. PLX038 was administered on day 1 and rucaparib after a gap of 3 days till day 19, in 21-day cycles. The patient was initially diagnosed with locally advanced invasive ductal carcinoma and was treated with neoadjuvant chemotherapy followed by surgery and radiation. Nine years later, she developed metastatic disease expressing estrogen and progesterone receptors, and was treated with multiple anti-endocrine therapies and chemotherapies including eribulin, abraxane, and carboplatin. The patient had a strong family history of cancer including a brother who died at age 37 of lung cancer, and breast cancer in a sister at age 48. At enrolment, she had skin lesions on the breast which were painful and pruritic and in addition had involvement of the lung and bones. Tumor and germline testing revealed an ATM p.Q1970* pathogenic mutation (c.5908C>T), located in coding exon 38. The mutation results from a C to T substitution at nucleotide position 5908 and creates a premature translational stop signal which is expected to result in an absence or disrupted ATM protein. The variant was not present in population databases such as ExaC but has been reported in the literature in individuals with ataxia-telangiectasia and recognized as a founder mutation in the Costa Rican population.

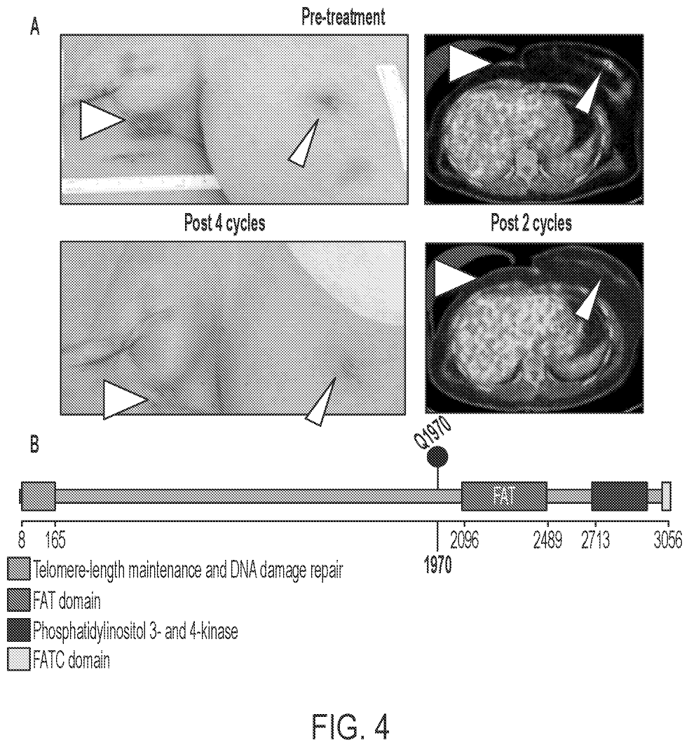

The combination of low-dose PLX038 and rucaparib resulted in substantial reduction in this patient's tumor burden and resolution of cancer symptoms within the first two cycles of treatment, qualifying as a complete response (FIG. 4). Shortly after initiation of treatment, the patient experienced non-dose limiting gastrointestinal adverse effects that required reduction of the rucaparib dose and then its suspension 4 months after treatment initiation. Thereafter, the patient was treated solely with single-agent, low-dose PLX038 that resulted in sustained suppression of the tumor which lasted for a total of 12 months after treatment initiation—a result consistent with the aforementioned high sensitivity of an ATM-deficient xenograft to low doses of PLX038A.

What is claimed is:

1. A method of treating cancer in a patient with an ATM-deficiency or ATR-deficiency, comprising administering to the patient a parenteral dose of PLX038 once every three weeks, wherein the dose provides a steady state AUC_{(0-∞)} of SN-38 from about 2,000 nM·h to about 8,000 nM·h.

2. The method of claim 1, wherein said dose provides a steady state AUC_{(0-∞)} of SN-38 from about 3,500 nM·h to about 7,500 nM·h.

3. The method of claim 1, wherein said dose provides a steady state AUC_{(0-∞)} of SN-38 from about 4,000 nM·h to about 7,000 nM·h.

4. The method of claim 1, wherein said dose provides a steady state AUC_{(0-∞)} of SN-38 from about 5,500 nM·h to about 6,600 nM·h.

5. The method of any one of claims 1-4, wherein said dose provides a steady state Cₘₐₓ of SN-38 of less than 100 nM.

6. The method of any one of claims 1-4, wherein said dose provides a steady state Cₘₐₓ of SN-38 of less than 80 nM.

7. The method of any one of claims 1-4, wherein said dose provides a steady state Cₘₐₓ of SN-38 of less than 40 nM.

8. The method of any one of claims 1-4, wherein said dose provides a steady state Cₘₐₓ of SN-38 of from about 30 nM to about 100 nM.

9. The method of any one of claims 1-4, wherein said dose provides a steady state Cₘₐₓ of SN-38 of from about 45 nM to about 85 nM.

10. The method of any one of claims 1-4, wherein said dose provides a steady state Cₘₐₓ of SN-38 of from about 50 nM to about 75 nM.

11. The method of any one of claims 1-10 wherein the dose of PLX038 once every three weeks is from about 350 mg/m² to about to about 2,000 mg/m².

12. The method of any one of claims 1-10, wherein the dose of PLX038 once every three weeks is from about 1,500 mg/m² to about to about 2,000 mg/m².

13. The method of any one of claims 1-10, wherein the dose of PLX038 once every three weeks is from about 1,500 mg/m² to about to about 1,800 mg/m².

14. The method of any one of claims 1-10, wherein the dose of PLX038 once every three weeks is about 1,730 mg/m².

15. The method of any one of claims 1-10, wherein the dose of PLX038 once every three weeks is about 1,000 mg/m².

16. The method of any one of claims 1-10, wherein the dose of PLX038 once every three weeks is about 1,100 mg/m².

17. The method of any one of claims 1-16, wherein the PLX038 is administered in combination with a PARP inhibitor.

18. The method of claim 17, wherein the PARP inhibitor is administered at least two days after the PLX038 is administered.

19. The method of claim 17, wherein the PARP inhibitor is administered four days after the PLX038 is administered.

20. The method of any one of claims 17-19, wherein the PARP inhibitor is rucaparib.

21. The method of claim 20, wherein the rucaparib is administered twice daily at a dose of from about 200 mg to about 400 mg.

22. The method of any one of claims 17-21, wherein the dose of PLX038 once every three weeks is from about 350 mg/m² to about to about 1,200 mg/m².

23. The method of any one of claims 17-21, wherein the dose of PLX038 once every three weeks is from about 750 mg/m² to about to about 1,100 mg/m².

24. The method of any one of claims 17-21, wherein the dose of PLX038 once every three weeks is from about 800 mg/m² to about to about 1,000 mg/m².

25. The method of any one of claims 17-21, wherein the dose of PLX038 is about 1,000 mg/m².

26. The method of any one of claims 17-21, wherein the dose of PLX038 is about 800 mg/m².

27. The method of any one of claims 1-26, where the PLX038 is administered intravenously.

28. A method of treating cancer in a patient with an ATM-deficient or ATR-deficient tumor, comprising administering to the patient a dose of PLX038 and a PARP inhibitor over a 3 week dosing schedule, wherein the PLX038 is administered parenterally on day 1 of the cycle and the PARP inhibitor is administered orally on days 5-19 of the cycle.

29. The method of claim 28, wherein the PLX038 is administered intravenously.

30. The method of claim 28 or claim 29, wherein the PARP inhibitor is administered twice daily.

31. The method of any one of claims 28-30, wherein the PARP inhibitor is rucaparib.

32. The method of any one of claims 28-31, wherein the PLX038 is administered at a dose of 1,300 mg/m².

33. The method of any one of claims 28-31, wherein the PLX038 is administered at a dose of 1,000 mg/m².

34. The method of any one of claims 28-31, wherein the PLX038 is administered at a dose of 850 mg/m².

35. The method of any one of claims 28-31, wherein the PLX038 is administered at a dose that provides a steady state AUC_{(0-∞)} of SN-38 from about 2,000 nM·h to about 8,000 nM·h.

36. The method of any one of claims 28-31, wherein the PLX038 is administered at a dose that provides a steady state AUC_{(0-∞)} of SN-38 from about 4,500 nM·h to about 7,000 nM·h.

37. The method of any one of claims 31-36, wherein the rucaparib is administered twice daily at a dose of 600 mg.

38. The method of any one of claims 31-36, wherein the rucaparib is administered twice daily at a dose of 400 mg.

39. The method of any one of claims 31-36, wherein the rucaparib is administered twice daily at a dose of 300 mg.

40. The method of any one of claims 28-39, wherein the patient has breast cancer.

41. The method of claim 40, wherein the patient has triple-negative breast cancer.

42. The method of any one of claims 28-39, wherein the patient has ovarian cancer.

43. The method of any one of claims 28-39, wherein the patient has small lung cancer.

44. A method of treating cancer in a patient in need thereof, comprising administering the patient a combination of PLX038 and an ATM kinase inhibitor or an ATR kinase inhibitor.

45. The method of claim 44, wherein the PLX038 is administered parenterally.

46. The method of claim 45, wherein the PLX038 is administered at a dose that provides a steady state AUC_{(0-∞)} of SN-38 from about 2,000 nM·h to about 8,000 nM·h.

47. The method of claim 46, wherein said dose provides a steady state AUC_{(0-∞)} of SN-38 from about 3,500 nM·h to about 7,500 nM·h.

48. The method of claim 46, wherein said dose provides a steady state AUC_{(0-∞)} of SN-38 from about 4,000 nM·h to about 7,000 nM·h.

49. The method of claim 46, wherein said dose provides a steady state AUC_{(0-∞)} of SN-38 from about 5,500 nM·h to about 6,600 nM·h.

50. The method of any one of claims 46-49, wherein said dose provides a steady state Cₘₐₓ of SN-38 of less than 100 nM.

51. The method of any one of claims 46-49, wherein said dose provides a steady state Cₘₐₓ of SN-38 of less than 80 nM.

52. The method of any one of claims 46-49, wherein said dose provides a steady state Cₘₐₓ of SN-38 of less than 40 nM.

53. The method of any one of claims 46-49, wherein said dose provides a steady state Cₘₐₓ of SN-38 of from about 30 nM to about 100 nM.

54. The method of any one of claims 46-49, wherein said dose provides a steady state Cₘₐₓ of SN-38 of from about 45 nM to about 85 nM.

55. The method of any one of claims 46-49, wherein said dose provides a steady state Cₘₐₓ of SN-38 of from about 50 nM to about 75 nM.

56. The method of any one of claims 44-55, wherein the dose of PLX038 once every three weeks is from about 800 mg/m² to about to about 2,000 mg/m².

57. The method of any one of claims 44-55, wherein the dose of PLX038 once every three weeks is from about 1,500 mg/m² to about to about 2,000 mg/m².

58. The method of any one of claims 44-55, wherein the dose of PLX038 once every three weeks is from about 1,500 mg/m² to about to about 1,800 mg/m².

59. The method of any one of claims 44-55, wherein the dose of PLX038 once every three weeks is about 1,730 mg/m².

60. The method of any one of claims 44-55, wherein the dose of PLX038 once every three weeks is about 1,000 mg/m².

61. The method of any one of claims 44-55, wherein the dose of PLX038 once every three weeks is about 1,100 mg/m².

62. The method of any one of claims 44-61, further comprising administering a PARP inhibitor.

63. The method of claim 62, wherein the PARP inhibitor is administered at least two days after the PLX038 is administered.

64. The method of claim 62, wherein the PARP inhibitor is administered four days after the PLX038 is administered.

65. The method of any one of claims 62-64, wherein the PARP inhibitor is rucaparib.

66. The method of claim 65, wherein the rucaparib is administered twice daily at a dose of from about 200 mg to about 400 mg.

67. The method of any one of claims 44-66, wherein the patient has breast cancer.

68. The method of claim 67, wherein the patient has triple-negative breast cancer.

69. The method of any one of claims 44-66, wherein the patient has ovarian cancer.

70. The method of any one of claims 44-66, wherein the patient has small lung cancer.

71. The method of any one of claims 44-70, wherein the cancer patient has a mutation in a gene that provides a protein that aids in DNA repair.

72. The method of claim 71, wherein the gene is a BRCA1 gene.

73. The method of claim 71, wherein the gene is a BRCA2 gene.

74. The method of any one of claims 44-73, wherein the ATM kinase inhibitor administered in combination with PLX038 is AZD0156, LY294002, KU-55933, or KU-59403.

75. The method of any one of claims 44-73, wherein the ATR kinase inhibitor administered in combination with PLX038 is AZD6738, M6620 (VX-970), BAY1895344 or M4344 (VX-803).
